(19) United States
(12) Patent Application Publication
(10) Pub. No.: US 2007/0208213 A1
(43) Pub. Date: Sep. 6, 2007

(54) Method and apparatus for in-vitro fertilization and tubal occlusion

(76) Inventor: Susan E. Swann, Grass Valley, CA (US)

Correspondence Address: BLAKELY SOKOLOFF TAYLOR & ZAFMAN, 1279 OAKMEAD PARKWAY, SUNNYVALE, CA, 94085-4040, US

(21) Appl. No.: 11/702,777

(22) Filed: Feb. 5, 2007

Related U.S. Application Data
(60) Provisional application No. 60/765,262, filed on Feb. 3, 2006.

(51) Int. Cl. A61B 17/43; A61D 7/00

(52) U.S. Cl. 600/33

(57) ABSTRACT

An apparatuses and methods to improve the effectiveness of in vitro fertilization are described. In one embodiment of a method, a patient suffers from infertility because of a tubal disease. The patient's ovaries are shut down, followed by over stimulation of the ovaries to produce at least one viable egg. After the at least one egg is harvested, a structure is placed in the diseased fallopian tube to functionally occlude the disease fallopian tube at least temporarily. Once the fallopian tube is occluded, in vitro fertilization of the harvested egg is performed. The fertilized eggs which become pre-embryos are subsequently implanted into the uterus of the patient.

This application claims the benefit of the filing date of U.S. provisional patent application Ser. No. 60/765,262, which was filed on Feb. 3, 2006. This provisional application is incorporated herein by reference.

### FIELD OF THE INVENTION

The invention relates generally to in-vitro fertilization and specifically, induction of tubal occlusion to alleviate complications that negatively affects success rates in in-vitro fertilization.

### BACKGROUND OF THE INVENTION

Infertility is the inability of a couple to become pregnant after 1 year of unprotected sexual intercourse. There are various different types of infertility, each may be caused by a problem in the male or female reproductive system. In the male, he may have semen abnormalities, including low sperm count or reduced sperm motility. There may also be sperm antibodies which reduce sperm numbers in the semen. The antibodies bind to the sperm and impair movement (motility), preventing the sperm from swimming the cervical mucus and reaching the egg. In the female, she may have damaged, blocked or absent fallopian tubes. Another cause in the female is endometriosis, a condition where the cells normally lining the uterus occur and grow in other parts of the body. This condition may lead to formation of scarring, adhesions and distortion of the reproductive organs thus interfering with the normal fertilization process. Still, there may be unexplained infertility, where no apparent cause is found for the infertility, despite thorough investigation.

A range of assisted reproductive technologies (ART) are typically available to couples to overcome the problem of infertility. These procedures all involve collecting the oocytes (eggs) and placing them in direct contact with the sperm. One such technique is in vitro fertilization Pre-embryo transfer (IVF-ET). This has been a known and widely accepted procedure as a solution to infertility and involves the uniting of egg and sperm in vitro (in the lab) and subsequently transferring the embryo(s) into the uterus through the cervix to allow pregnancy to begin. There are other ARTs as well, such as gamete (egg and sperm) intrafallopian transfer (GIFT) and zygote intrafallopian transfer (ZIFT), but both of these procedures require minimal invasive laparoscopic surgery involving general anesthesia and a small incision. The former requires laparoscopy to transfer the gametes, while the latter requires laparoscopy to transfer embryos through the cervix. Since IVF does not require surgery and provides the same success rates as ZIFT, it has supplanted both GIFT and ZIFT.

A typical in vitro fertilization (IVF) cycle begins with shutting down the ovaries with a medication. Next phase involves stimulation of the ovaries with potent ovulation medications. When the eggs are ready for harvesting, a final step is to induce final maturation in the eggs. The eggs are then harvested by a process called ultrasound guided vaginal retrieval. Under heavy sedation and with ultrasound guidance, a thin needle is passed a short distance into the ovaries and the eggs are suctioned from the follicles. Typically, 5-15 eggs are collected and fertilized by adding approximately 100,000 motile sperm to each egg. If the sperm will not fertilize the eggs naturally, intracytoplasmic sperm injection (ICSI) is performed by manually puncturing the egg directly under a microscope and injecting one sperm in the egg. After fertilization, and when the cells divide 2 or 3 times to become preimplantation embryos (pre-embryos), the pre-embryos will be passed through the vagina and into the uterus using a special catheter.

While IVF is a solution to most infertility problems experienced by couples, IVF success rates can be negatively impacted by tubal disease such as hydrosalpinx, and other conditions such as endometriosis. Hydrosalpinx is a condition associated with a blocked, dilated, fluid-filled fallopian tube caused by a previous tubal infection. Endometriosis, as explained briefly, may interfere with IVF if the abnormal uterus lining cells grow into the fallopian tube forming adhesions and distortions. For some women who do not have hydrosalpinx, or endometriosis, they may have only a single diseased fallopian tube and one other healthy fallopian tube to allow for natural pregnancy.

### SUMMARY

At least certain embodiments of the invention are designed to resolve the issues presented by hydrosalpinx and/or other tubal diseases, thereby increasing IVF success rates. At least certain embodiments of the invention may also be an alternative to women with a single healthy fallopian tube to allow for natural pregnancy when traditional tubal ligation of the diseased fallopian tube via laparoscopy was the only option. Tubal ligation is performed under general anesthesia using a trocar to access the patient's peritoneal cavity and carries a risk of death or injury.

A methods and apparatuses to improve in vitro fertilization (IVF) are described. In one embodiment, a patient's ovaries are shut down, the ovaries are stimulated to produce multiple eggs (e.g. oocytes) in the ovary, multiple eggs are harvested from the ovaries for in vitro fertilization, a structure is placed (e.g. a structure is deployed from a delivery catheter or formed in situ) in the fallopian tube to cause, for example, in growth of tissue in the fallopian tubes while the patient is being treated for her infertility, and before or after the fallopian tubes are occluded, the fertilized pre-embryo is implanted into the uterus.

In one embodiment, a structure may be a device, such as the Essure device from Conceptus, Inc. of Mountain View, Calif., and it can be used to occlude one or both fallopian tubes in a patient in order to, for example, prevent accumulated fluid in a hydrosalpinx condition from reducing the success of an embryo implantation after in vitro fertilization. In other cases, a structure may be used to occlude one or both fallopian tubes for a patient who has a diseased fallopian tube or who suffers from endometriosis in order to improve the chances for in vitro fertilization.

Other methods and other apparatuses are also described.

### DESCRIPTION OF THE DRAWINGS

The present invention is illustrated by way of example, and not limitation, figures of the accompanying drawings in which:

FIG. 1A illustrates a healthy female reproductive system.

FIG. 1B illustrates a female reproductive system with diseased fallopian tubes.

FIG. 2 illustrates a flow chart of a combination procedure of in vitro fertilization and fallopian tube occlusion.

FIG. 3 illustrates a flow chart of alternate steps of a combination procedure of fertilization and fallopian tube occlusion.

FIG. 4A illustrates access of a delivery device into the fallopian tube.

FIG. 4B illustrates placement of a structure inside the fallopian tube.

### DETAILED DESCRIPTION

In the following section, several embodiments of, for example, processes, devices and methods are described in order to thoroughly detail various embodiments. It will be understood by one skilled in the art that practicing the various embodiments do not require the employment of all or even some of the specific details outlined therein. In some cases, well known methods or components have not been included in the description in order to prevent unnecessarily masking various embodiments.

The present invention provides intrafallopian devices and methods for their placement into fallopian tubes in combination with in vitro fertilization techniques, such as guided IVF, to increase in vitro fertilization (IVF) success rates for patients suffering from infertility.

IVF is a process of removing a mature ovum from the stimulated ovary, combining the ovum with sperm outside of the body, allowing the fertilized ovum to grow and divide, and then replacing the ovum transcervically into the uterine cavity with hopes of implantation and establishment of pregnancy. The primary indications of IVF are tubal disease, oligospermia, endometriosis and unexplained infertility. Although IVF is a typically successful alternative for couples to overcome the above problems to conceive, the success rate of the pregnancy may be still be negatively affected after a fertilized pre-embryo or fertilized ovum is implanted into the female who suffers from tubal disease and/or endometriosis. The use of intrafallopian devices to occlude the fallopian tubes is an approach to increase success rates of IVF by protecting the pre-embryo or fertilized ovum implanted in the uterine tissue beneath the surface of the endometrial lining. The device indirectly acts to protect the embryo from any toxic fluid which may flow into the uterine cavity and threaten the embryo. Moreover, the device acts to directly protect the implanted embryo from accidentally being carried into the fallopian tubes by uterine peristaltic movements which may result in tubal gestation following IVF.

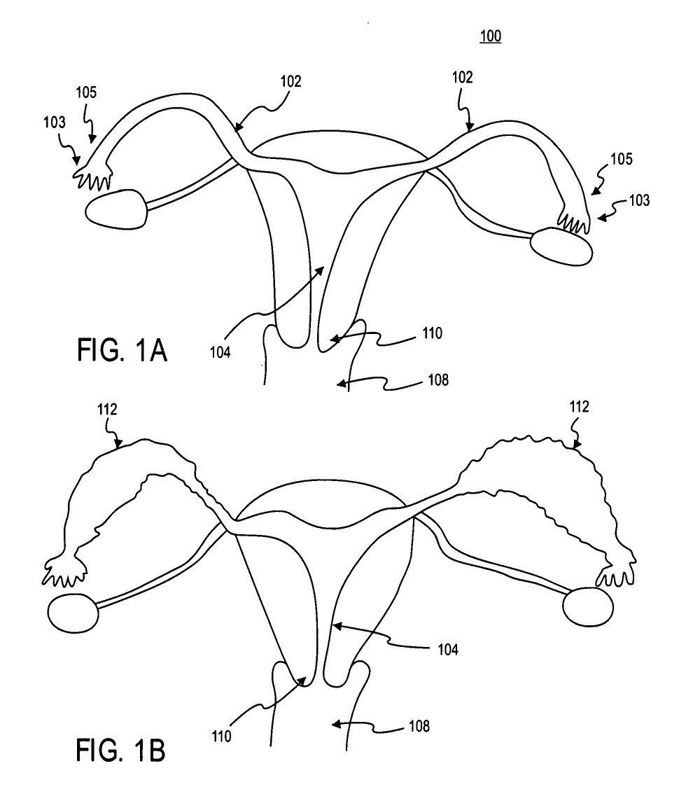

FIGS. 1A and 1B illustrate the difference in a healthy and a diseased fallopian tube in the female reproductive system. The effects of a diseased fallopian tube on IVF will be explained. FIG. 1A illustrates a female reproductive system with healthy fallopian tubes. Typically, the oocyte is developed within the ovary and released from the ovary during ovulation and proceeds through the oviduct. The distal fingers (fimbria 103) of the fallopian tube embrace the ovum and envelope it in the distal tube, the ampulla 105. Under normal conditions, fertilization only occurs in the ampulla 105, where the sperm meets and unites with the ovum. The sperm reaches the ampulla in a journey which started in the vagina 108 and subsequently proceeding through the cervical os 110, the uterus 104, and the fallopian tube 102. After fertilization, the zygote (fertilized ovum) slowly migrates down the fallopian tube and attaches to the endometrium in the uterus. The zygote is nourished and develops into an embryo.

FIG. 1B illustrates a female reproductive system with a diseased fallopian tube. As illustrated, the enlarged and sausage shaped swelling resulting from an inflamed fallopian tube can be a result of infection or injury to the fimbria, ampulla or the fallopian tube. Classic causes are chalmydia and gonorrhea or other sexually transmitted diseases and may even be caused by endometriosis, where there is abnormal growth of the cells lining the uterus and the fallopian tubes and other parts of the body. Consequently to the injury or disease, inflammation takes place, the glands within the tube produces a watery fluid that collects in the tube(s) and the fallopian tube(s) is/are enlarged as illustrated. Often, the fimbria themselves are injured. They are fused together and become ineffective in searching and moving the egg into the fallopian tubes. Further, the delicate cells that contain actively moving hair-like cilia in the fallopian tube are lost. Fluid collects within the closed tube and the condition of hydrosalpinx is manifested.

Hydrosalpinx causes infertility and reduces the success rates of fertility treatment, including those treatments that bypass the fallopian tubes. The blocked tubes can communicate with the uterus, and the fluid in the tubes can be expressed out of the tubes into the uterus. The fluid is toxic to early embryo development and does not provide a favorable environment to an implanted embryo. Fertility drugs may cause the fluid to build up in the tube, since the tubes are responsive to the ovarian hormones produced during fertility drug therapy. The large volume of the fluid that flows back into the uterus can also produce enough flow that the artificially implanted embryos (through the IVF procedure) find it difficult to attach since they have no ability to move against the tide.

Hydrosalpinx can be repaired in certain selected cases, but pregnancy rates remain rather low. A laparoscopic procedure known as neosalpingostomy may be performed to make an incision to open up the tubes, but the tube often closes back up and hydrosalpinx has a high recurrence rate. Often, a small hydrosalpinx with mild damage to the fallopian tubes is the most successfully repaired. The prognosis for future fertility after surgical repair of the damaged fallopian tubes thus depends heavily on the extent of damage that has accumulated prior to surgical repair and the microsurgical techniques used in the repair of the tubes. Pregnancy often requires 6 months to a year after surgery and is suitable for younger women with relatively healthy ovaries and eggs and time. Some young women choose tubal reanastomosis, but it is rarely an option for women in their thirties or those with other factors contributing to their infertility. Further, tubal surgery can cause scarring, which can impede egg transport, even when performed by the most skilled surgeons. Older women with a large hydrosalpinx do not benefit from surgical repair and the only option left for them is tubal removal through laparoscopic salpingectomy. The cost of surgical tubal ligation ranges up to $6000 and carries a risk of serious complications and even death. Whereas, the cost of a device or structure as described to occlude the fallopian tubes which can also resolve the same issues as surgery, ranges up to only $3000. Therefore, comparing the two approaches, there is also an added benefit in cost savings in the use of a device to achieve tubal occlusion compared to that of the surgical alternative.

Other issues causing fertility problems may result from prior abdominal surgery, other tubal issues, and pelvic inflammatory disease (PID). Prior abdominal surgery can cause adhesions. Extratubal adhesions may distort the normal course of the tube within the pelvis thus interfering with the motility of sperm into the fallopian tubes to fertilize the egg or for the transport of the egg into the uterus. Similarly, other tubal issues such as destruction of the delicate tissues and folds that line the inside of the fallopian tubes (the mucosa and rugae), extratubal adhesions, fimbrial agglutinations that obstructs the ability of the fallopian tube to capture and transport an egg, directly leads to infertility. PID involving the reproductive organs can rapidly destroy the reproduction function of the organs. Typically, infectious or chronic inflammatory process involving the adnexae (ovaries and fallopian tubes) initially result in distal uterus obstruction of the tube and damage to the tissues within the tube, leading to infertility. Women with laparoscopically confirmed PID suffer from tubal factor infertility rapidly. The risk of infertility following an episode of PID was seen to relate to the woman's age, number of infections, and the severity of infection. There is generally a six to ten fold increase in the ectopic pregnancy rate following an episode of PID.

Yet another cause of infertility can be attributed to proximal tubal blockage. Proximal tubal obstruction can be caused by previous pelvic infection, a thicknening and inflammation of the tubal wall, also known as salpingitis isthmica nodosa (SIN), mucus plugs, or endometriosis. The diameter of the fallopian tube is quite small within the uterine wall but increases distally towards the opening near the ovary. The “spasm” of uterine muscles during the HSG may constrict or occlude one or both of the fallopian tubes. Small plugs of material, usually thought to be mucus or proteinaceous debris, can therefore easily block the proximal tube(s) where it is very narrow within the uterus. SIN refers to nodal scarring of the tubes. Specifically, it refers to the nodular appearance of the inflammation of the tubes in the isthmica region near the top of the uterus. SIN is manifested by large nodules trapping old blood, expanding the nodular area and prevents a clear pathway for the sperms to travel. Further, SIN also increases the chance of an ectopic/tubal pregnancy due to the internal scar in the tubal canal that transports the egg to the uterus. The scarring simply prevents the fertilized egg from being moved into the uterus so the egg matures in the tube instead.

Endometriosis occurs when the endometrial tissue lining the inside of the uterus covers parts of the outer tissue (Serosa) of the uterus, the colon, the abdominal lining and the bladder. Overtime, scarring may show up in and around the pelvis, ovaries, and fallopian tubes. The scarring around the ovaries and the fallopian tubes can prevent eggs that are released from the ovaries to make it into the fallopian tubes, leading to infertility or inability to get pregnant. This can be caused by pieces of tissue flow back into the fallopian tubes instead of flowing outside of the body through the vagina during the menstrual period, flows. Permanent occlusion of the fallopian tubes will leave the patient with only the option of IVF to become pregnant, but may also remove many other complications and diseases as described.

The combination of IVF and occlusion of the fallopian tubes present a minimally invasive, low risk, non-surgical option to patients who suffer from tubal disease such as hydrosalpinx and/or other issues that causes infertility as described above. The additional procedure of fallopian tube occlusion increases the success rate of the IVF procedure by reducing the complications that may occur to the implanted embryo after in vitro fertilization.

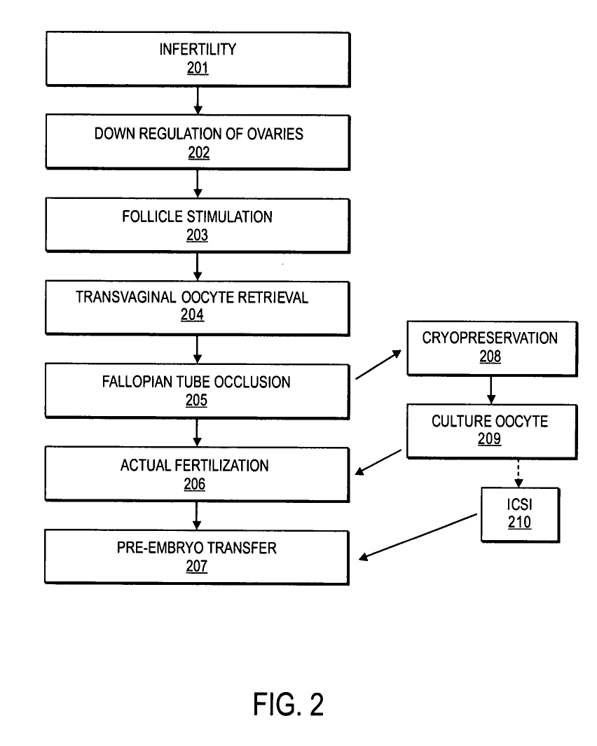

Methods of in vitro fertilization with the tubal occlusion device will now be described. FIG. 2 is a flow chart that illustrates the combination procedure of IVF and the use of an occlusion device to occlude the fallopian tubes when eggs are harvested through the fallopian tubes. In box 201, a patient is diagnosed with an infertility problem. Infertility may be caused by tubal disease, oligospermia, endometriosis or other unexplained reasons. In vitro fertilization is initiated with down regulation of the ovaries in box 202. The IVF cycle may begin with shutting down the ovaries by using a hormone to prevent the pituitary from normal stimulatory function. The hormone is often a GnRH (Gonadotropin Releasing Hormone) agonist to overstimulate the pituitary gland so that it is overloaded and shuts down, thus stopping the production of luteinizing hormone (LH) and follicle stimulation (FSH) which are responsible for ovulation. The most commonly used drug is Lupron. Lupron is given approximately two weeks after which the ovaries shut down completely. Other choice of drug to use is an GnRH antagonist, such as Cetrorelix, which directly shuts down the pituitary gland by blocking the pituitary gland's use of GnRH. GnRH agonists works in a few days, while GnRH antagonists work in a few hours.

The next phase may be follicle stimulation as in box 203. Normally, the ovaries produces only one egg in each ovulation cycle. The object of this phase is to stimulate the ovaries to produce multiple eggs. This phase typically lasts for approximately two weeks and the patient receives daily injections of ovary stimulation hormones. Pergonal is a common drug used to stimulate follicle development. The final process in follicle stimulation development is to give human chrionic gonadotropin (hcG) to induce final maturation in the eggs.

When the eggs are ready to be harvested, a procedure known as Transvaginal oocyte retrieval, box 204, is used to retrieve the eggs. When the fallopian tubes are still relatively patent but the environment is not favorable for a sperm to travel or for an egg to develop, one may utilize an image guided system such as fiber optic or ultrasound guided probe with a needle guide to traverse the vagina, cervix, uterus and entire length of the fallopian tube into the ovary to target the follicles and vacuum aspirate the eggs for in vitro fertilization. This discussion assumes that multiple eggs are harvested and that multiple eggs may even be fertilized with sperm. It will be understood that, in other embodiments, a single egg, rather than multiple eggs, may be harvested and a single harvested egg may be fertilized to create a fertilized ovum.

Once multiple eggs are harvested, multiple eggs are cultured, as in box 208. The best cultured eggs are then selected and cryopreserved, as in box 209. The rationale is that by cryopreserving the eggs, fertilization can be delayed in light of the delayed fertilization which is necessary to allow for fallopian tube closure before implantation of the in vitro fertilized pre-embryo or fertilized ovum into the uterine lining (in those embodiments in which it is desirable to occlude the fallopian tube before implanting the fertilized ovum into the uterus). This becomes necessary if the doctor opted for the egg harvesting procedure through the fallopian tubes. Typically, cryopreserving eggs 208 can also allow the patient to undergo one or more future IVFs without having to undergo another round of hormone treatments involving inhibition of ovary activity, follicle stimulation and egg harvesting procedure. When the fallopian tube closure is complete, multiple eggs are cultured, as in box 209, where the best eggs are selected and prepared for an in vitro fertilization process.

However, before fertilization of the eggs and implantation of the pre-embryo or fertilized ovum, the patient may be treated for the disease which causes infertility. In one embodiment where a patient suffers from tubal disease such as hydrosalpinx, fallopian tube occlusion, box 205, which may use a structure which induces tissue in growth in the fallopian tubes, is a minimally invasive alternative to that of a tubectomy or salpingectomy or the removal of the fallopian tubes. With the insertion or placement of these structures (details of the insertion and the structure itself is further described below), scar tissue may be caused to grow and to block the opening of the tubes. This is a safer alternative compared to surgical salpingectomy or salpingotomy which carries risks of complications and death. There is no need for general anesthesia, no need for a surgical suite or hospital setting to perform the procedure and no incisions are necessary. The procedure also allows the patient to recover faster. Normally, it takes approximately anywhere from zero to six months after insertion of the microstructures or microinserts for the tubes to be permanently blocked depending on the choice of device used. In one embodiment, a device can provide immediate physical blockage of the fallopian tubes. In another embodiment, a device can be inserted to induce tissue growth and may take up to six months for a complete occlusion by tissue growth. Further, a combination device can be used which provide physical blockage as well as tissue in growth.

A hysterosalpingogram (HSG), where x-ray is used to visualize the uterus and the fallopian tubes after a dye has been injected through the cervix, may be used to determine the degree of blockage in the fallopian tubes. Other methods to verify tubal occlusion may include saline ultrasound, and/or contrast infused sonography (CIS), a method using microbubbles in a medium to increase ultrasound visibility and detect fluid flow down a fallopian tube, and even laparoscopy. In at least certain embodiments, patients being treated for infertility due to problems in the female reproductive system such as tubal diseases, endometriosis, etc., should not be implanted with the in vitro fertilized ovum until at least a diseased fallopian tube has been functionally occluded or otherwise treated. Waiting, in these certain embodiments, for complete closure of the fallopian tube will ensure there is no leakage of any toxic fluids into the uterus from the fallopian tubes because of hydrosalpinx or other infections to threaten the environment of the pre-embryo. Further, complete closure of the fallopian tubes can also prevent the pre-embryo from being carried into the fallopian tubes which results in ectopic tubal gestation leading to ectopic pregnancy.

After insertion of the structure to induce tissue growth in the fallopian tubes, actual fertilization, box 206, may take place. Fertilization should be done shortly before anticipated implantation of the pre-embryo. Typically, the eggs selected are fertilized by adding approximately 100,000 motile sperm from a donor to each egg. Typically, the donor of the sperm is the spouse or partner of the patient. If the sperm will not fertilize the eggs, intracytoplasmic sperm injection (ICSI), box 210, can be performed. This is a manual procedure of puncturing the egg directly under a microscope and directly injecting a sperm into the egg to carry out fertilization. Often, multiple eggs are retrieved and more than one egg is fertilized to increase the chances of one fertilized egg developing into an embryo. However physicians may also only retrieve one egg and fertilize only one egg for development into an implantable pre-embryo.

Alternatively, couples may select to use either donor eggs from a different female or donor sperms from a different male or both. In the case of using donor eggs, the patient may not need to receive Lupron or similar drugs to shut down the ovaries and receive pergonal or similar drugs to stimulate the ovaries to produce multiple eggs. Rather, a patient may simply elect to have the structure inserted into the fallopian tubes to block the tubes and/or induce tubal occlusion. Also, donor sperms may be used in place of the patient partner's sperm if the patient's partner either has immotile sperms or if the partner is a female. The use of donor sperms and/or donor eggs do not change the inherent process, it merely suggests a different source of genetic materials to be used for the in vitro fertilization process.

After the one or eggs have been fertilized and become pre-embryos or at least one fertilized ovum, they are ready for cryopreservation or implantation into the uterus. Once the zygotes are allowed to grow and divide for 24, 48 or 72 hours, pre-embryo transfer can take place, box 207. Pre-embryos are generally withdrawn into a catheter with a non-stick surface, for example, coated with polytetrafluoroethylene (PTFE) or TEFLON™. The catheter is passed through the cervical os into the endometrial cavity. The pre-embryo and the medium in which they are contained in the catheter are then discharged into the endometrial cavity. Preferably, the catheter is placed directly into the uterine tissue depositing the pre-embryo within the endometrial lining so that they are positioned securely. The depth at which the embryo is deposited is typically from about 0.5 mm to about 5 mm beneath the endometrial surface, preferably, the pre-embryo is placed at a depth of about 0.5 mm to about 2.0 mm and most preferably at about 1.0 mm depth.

A pre-embryo or fertilized ovum from in vitro fertilization is merely placed in the uterine tissue (as described below). The pre-embryo or fertilized ovum does not have the benefit of a naturally conceived embryo which firmly plants or anchors itself in the uterine tissue when the fertilized egg is moved naturally from the fallopian tube into the uterus. Therefore, a pre-embryo or fertilized ovum from in vitro fertilization is susceptible to the peristaltic movement of the uterine tissue or the waves of fluids which can easily carry the pre-embryo or fertilized ovum into an open fallopian tube. Through the use of this structure, or a structure similar to that of a stent, to completely block the fallopian tubes and/or to induce tissue growth resulting in occlusion, there is an added benefit of minimizing any risk of ectopic pregnancy in the fallopian tubes when the pre-embryo or fertilized ovum is not firmly anchored in the uterine tissue and lacks the stability to resist movement..

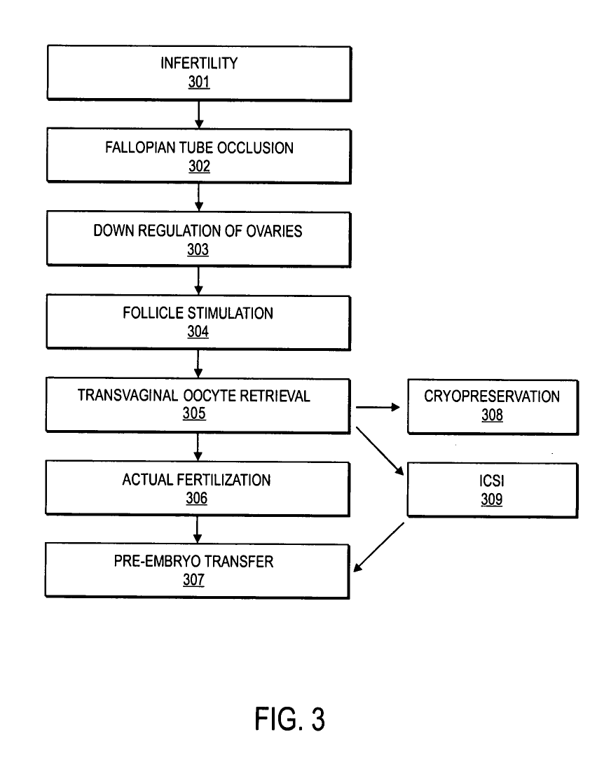

Properly placing the pre-embryo in a secured location is often essential to the success of the pregnancy. Since a pre-embryo or fertilized ovum is relatively mobile following transfer, the non-stick surface of the catheter is to prevent the pre-embryos from sticking to the catheter and ensure that the pre-embryo or fertilized ovum is deposited in the uterine tissue. Further, contractions and peristalsis of the uterine musculature produce movement of intrauterine fluids, thus, an image guided system is best used to assist in placement of the pre-embryos by visually selecting a location for placement. For example, a flexible fiberoptic hysteroscope or endoscope attached to a guide that is capable of detecting depth of penetration is ideal. A guide or open channel receives the microcatheter containing the pre-embryo or fertilized ovum and directs the microcatheter to the desired location by the image guided system for placing the pre-embryo or fertilized ovum at the optimum location. Another means of visualization such as ultrasound or x-ray can be used. It will be understood that the method shown in FIG. 2 is one of many possible embodiments and that, in other embodiments, the operations of FIG. 2 may be performed in a different order or some operations may be omitted or additional operations may also be performed. For example, in certain alternative embodiments, a fallopian tube occlusion device may be placed in one or more diseased fallopian tubes after harvesting at least one egg and after implanting a fertilized ovum. In other alternative embodiments, a fallopian tube occlusion device may be placed before harvesting at least one egg and before implanting of a fertilized ovum. FIG. 3 shows an example of such an alternative embodiment.

An alternative method of in vitro fertilization in combination with using an occlusion device to treat tubal disease is described in FIG. 3. FIG. 3 shows the same method as FIG. 2 with the exception that the eggs are harvested with a sterile procedure by-passing the fallopian tubes. This procedure is different from FIG. 2 in that the fallopian tubes are either so completely filled with fluid where access to the ovaries to retrieve the eggs through the fallopian tubes is impossible or where a salpingectomy is required to treat the disease. In any case, this procedure may be used when the eggs cannot be harvested with access from the fallopian tubes.

When the fallopian tubes are in such poor condition, the physician can opt to perform the tubal occlusion procedure first by inserting a structure such as a microstructure or microinserts into the fallopian tube first as in box 302, after diagnosing the infertility causes, box 301. While waiting for the occlusion inside the fallopian tube, effectively achieving tubectomy, disconnecting the uterus from the ovaries, the typical in vitro fertilization process can be initiated. For example, the down regulation of ovaries, box 303, follicle stimulation, box 304, can both be initiated a period of time after the structure is inserted. Ideally, they should be timed such that the transvaginal oocyte retrieval, box 305, and the subsequent fertilization, box 306, and pre-embryo transfer, box 307, can be performed near the end of period of time required for either functional occlusion or other treatment relating to a reproductive disease.

In this case, an alternate procedure to retrieve the eggs may be used, relying on a needle to penetrate the ovaries in the abdominal cavity of the patient guided by ultrasound probe (or other known techniques). Specifically, the ultrasound probe is advanced into the vaginal cavity and the needle guide serves as a passageway for the introduction of an aspirating and flushing needle (or other suitable) instruments. When the probe and the guide are inserted deeply into the vaginal vault, the aspirating needle is introduced into the needle guide. Thereafter, when advanced, the needle need only penetrate the thin vaginal wall to gain access to the ovary located inside the abdominal cavity. The ultrasound transducer in the distal end of the vaginal probe makes possible the projection of a clear image of abdominal organs onto a nearby ultrasound screen. Direction and depth of the aspirating needle can be observed on the ultrasound screen and directed to the target ovarian follicle where vacuum aspiration of the egg-containing follicle is a relatively safe and simple maneuver. In other words, the ultrasound-guided transvaginal placement of a needle beneath the cervix in the vagina enters the peritoneal cavity and sequentially drains each follicle to isolate its ovum.

After retrieval of the eggs, actual fertilization, box 306, or ICSI, box 307, both described above, can take place immediately. Cryopreservation, box 308, is only necessary if a patient wants to save her eggs, or to preserve the fertilized zygotes for future IVF.

The fallopian occlusion device and delivery will now be described. The fallopian occlusion device structure used in a fallopian tube may be delivered transcervically into the fallopian tube and may be anchored within the fallopian tube. The structure can be fully advanced into the fallopian tube or only partially advanced into the fallopian tube. The structure may provide blockage of the uterotubal junction to achieve effectiveness.

The structure used in this application can take on many different forms. Applicant hereby incorporates by reference a device and method to provide long term contraception or permanent sterilization similar to that described in U.S. Pat. No. 6,705,323 and further incorporates by reference the delivery system used to deliver a similar device also for the purpose of long term contraception as described in U.S. Pat. No. 6,709,667 B1. Both patents are commonly assigned to the same owner as this application and are incorporated herein by reference. Further, other devices and methods for fallopian tube occlusion are described in U.S. Pat. Nos. 6,432,116, and 5,095,917 and 6,096,052 and 6,346,102, all of which are incorporated herein by reference. A structure may be fabricated to include metal or plastic or other materials or a combination of different materials. It may be formed in situ in a portion of the fallopian tube (e.g., it may be formed out of a hydrogel which is injected into the fallopian tube). It may include an adhesive to secure it to a wall of a fallopian tube. A structure in certain embodiments may be fabricated to have a physical size which is designed to forcibly engage the walls of a fallopian tube that is enlarged by a disease; this physical size may be selected so that the structure can securely fit into fallopian tubes having different diameters. A structure in certain embodiments may be configured to elute at least one drug which may increase a fibrotic response (e.g., to promote tissue ingrowth to occlude a fallopian tube) or to treat a disease which, if not treated, may reduce chances for a successful IVF; for example, a structure may elute (from, for example, depots or a layer on the structure) a drug to treat a disease of a fallopian tube (such as, for example, hydrosalpinx, or endometroiosis, or fibroids, etc.). The structure may be placed in a fallopian tube which has been or is to be exposed to energy (e.g. electrical or heat energy) or one or more chemicals to cause scar tissue to from near or around the structure to occlude the fallopian tube. A structure may in certain embodiments, be a porous structure to allow tissue ingrowth into the structure, although tissue ingrowth is not required in all embodiments. A structure may resemble a single wall stent or a set of coils (e.g., an inner and outer coil, such as an Essure device) or a plug or a resorbable plug or a biodegradable plug or device. In certain embodiments, the structure may be removable (e.g. through the operation of a transcervical catheter) or degradable over a period of time to allow the completion of a successful pregnancy; hence, temporary occlusion or treatment (without occlusion) of a fallopian tube disease, or other disease which negatively effects IVF, may be all that is needed rather than permanent occlusion. A structure may be placed in a fallopian tube with an imaging technique that allows an operator to verify proper placement of the structure; for example, placement may be verified with x-ray imaging or ultrasound imaging or endoscopic imaging or magnetic resonance imaging. A structure may have a trailing material, which is visible in the uterus, after a proper placement if the trailing material does not effect fetal development. In those embodiments in which occlusion of a fallopian tube is desired, the occlusion may be verified before implanting a fertilized ovum and may be verified by using ultrasound imaging or x-ray imaging or other methods known in the art.

In one embodiment, the structure is shaped like a straight coil where it can be axially compressed and thus expand in length when not compressed. The anchoring of a straight coil is aided by the axial curvature of the tortuous fallopian tube. A further modification of this structure would be to increase the radius of curvature of the coil in the proximal and distal region to ensure anchoring of the structure to the tubal wall. While the fallopian tube does not have a constant inner diameter along its length, there can be a varying radii of curvature along the structure to improve anchoring and ensure maximum contact with tubal wall.

In another embodiment, the current structure has a lumen-traversing region with a helical outer surface. The helical surface is mechanically anchored by a resilient portion of the structure which is biased to form an enlarged secondary shape, preferably forming distal and proximal loops to anchor the structure into the lumen and ostium of the fallopian tube respectively. The structure itself can be formed from a coil of tightly wound metallic filament, with the coil further forming the helical outer surface and the secondary shapes of the distal and proximal loops as described. A modification of this embodiment is where the lumen traversing region has a helical outer surface and a cross-section which is smaller than the cross-sections of the proximal and distal anchors. Further, the lumen traversing region comprises a resilient structure, generally having a ribbon wound over the outer surface to form the helical shape. Anchoring can further be enhanced by a sharp outer edge on the ribbon.

Yet another embodiment comprises a primary coil having a proximal loop, a distal loop, and an intermediate straight section between the loops. A helical ribbon is wound over at least a portion of the intermediate section, forming a helical surface to mechanically anchor the device within the fallopian tube. An element is disposed along the coil, and is adapted to incite a tissue reaction in the tubal tissues. The ribbon has an approximate width in the range between about 0.005 in and 0.1 in, an approximate thickness in the range between about 0.001 in and about 0.2 in, and an approximate pitch in the range of between about 0.01 in and 0.2 in. The overall device geometry preferably facilitates introduction and retention but is not large or rigid enough to interfere with internal tissue movements. The structure's length approximately ranges between about 1.5 cm and 15 cm when in relaxed state, while the distal and proximal loops have outer diameters of at least 3.0 mm. The primary coil approximately has an outer diameter in the range between 0.2 mm and 5.0 mm.

In another embodiment, the structure can have an elongated coil which is substantially straight. Such straight coils or cylindrical structures or substantially cylindrical structures are positioned axially within the tortuous fallopian tubes and the bends imposed on the coil by the fallopian tube can result in resilient anchoring of the coil. The straight resilient coil can act as an integral guidewire during transcervical deployment of the device within the fallopian tube.

In another embodiment, the structure can take on the shape of a meshed cylindrical tubing, much like that of a stent. The structure can be made of any type of resilient metal including stainless steel, nickel titanium or a resilient polymer. Rather than the object of keeping the tubal lumen patent, the pattern of the structure will be cut to induce tissue growth. The structure can be balloon expandable or self-expandable.

Yet in another embodiment, the structure expands like the frame of an umbrella and forms a similar shape as an umbrella. Further, if viewed in a cross-sectional view of the fallopian tube, it may appear similar to that of a spider web with cross members extending radially from the center of the device towards the walls of the fallopian tube and having cross-members intersecting and connecting these radial elements extending outward. Also, this structure, acting similar to that of a plug, can be implanted after radiofrequency energy is used to ablate the tissue, causing or inducing scarring. Similarly, the radiofrequency energy may be transmitted via the structure or device which is used as an electrode.

In all the embodiments described above, the structure may or may not extend from the fallopian tube(s) into the uterine cavity. In the case if the structure does extend from the fallopian tube(s) into the uterine cavity, the portion extended into the uterine cavity is designed to not affect blastocyst implantation and cause any problem or issue with the development of the embryo or affect the overall pregnancy of the patient.

In additional to the embodiments of the structure as described above, there are other aspects that can be combined with any of the embodiments in this disclosure to enhance effectiveness of the structure to induce tissue growth. In one embodiment, copper is used as the material for the ribbon or the coil. Copper inherently elicits tissue growth. Thus the resilient structure of a metallic coil can include a copper alloy or plating, ideally comprising at least 75% copper. In another embodiment, tissue reaction may be incited within the fallopian tube using a coating, a surface treatment, or any mechanical interaction between the structure and the surrounding tubal wall. For example, an element disposed along the primary coil may include a braided or woven polyester fiber such as Dacron®, Rayon®, a micro-porous material or surface treatment. Alternatively, a sharp edged helical ribbon or other mechanical interaction may incite the formation of scar tissue or a surface coating of the coil may sclerose the tubal tissue exciting formation of fibrous connective tissue. Furthermore, drugs which can be used to induce tissue growth can be coated over the structure.

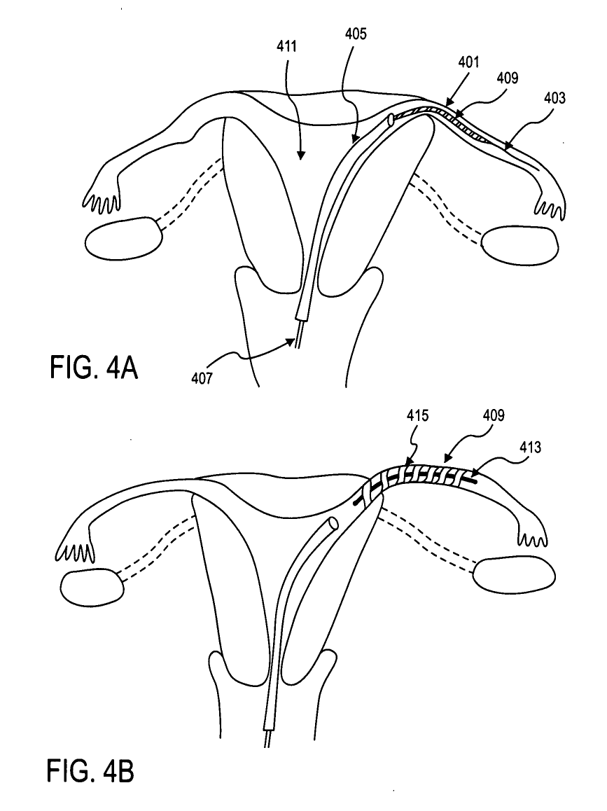

The delivery of a structure is now described. FIG. 4A shows the access of a structure delivery device positioned in the fallopian tube. The entire access and delivery of the structure may be performed under the assistance of x-ray equipment, ultrasound probe or fiber-optic imaging device or other imaging methods. A guide wire 403 is initially used to access the fallopian tube 403. A catheter 405 carrying the delivery system 407 of the structure 409 is then advanced into the fallopian tubes together. After the structure 409 and the delivery device 407 is positioned in the fallopian tube, the catheter is withdrawn proximally into the uterine cavity 411 leaving the structure 409, which includes an outer coil 415 and an inner coil 413 in this embodiment, inside the fallopian tube. FIG. 4B shows the structure 409 after it has been deployed. The structure may be inflated using an inflatable balloon, but is preferably self-expanding. Thus on the delivery catheter device, there is a sheath which prevents the structure from deployment. Only when the sheath is retracted proximally after the target position is reached will the structure expand and deploy. The structure shown in FIG. 4B has a primary coil 413 and a helical structure 415 as described in an earlier embodiment.

In the foregoing specification, the invention has been described with reference to specific exemplary embodiments thereof. It will, however, be evident that various modifications and changes may be made thereto without departing from the broader spirit and scope of the invention as set forth in the appended claims. The specification and drawings are, accordingly, to be regarded in an illustrative rather than a restrictive sense.

What is claimed is:

1. A method for in vitro fertilization comprising:
stimulating growth of follicles to produce at least one viable egg in an ovary;
harvesting at least one egg from the ovary of a patient;
placing a structure into a fallopian tube of the patient, the structure being configured to functionally occlude the fallopian tube at least temporarily;
occluding the fallopian tube in which the structure is placed to prevent any transfer of fluid between the tube and the uterine cavity;
fertilizing an egg, obtained from the harvesting, in vitro with a sperm from a donor; and
implanting a pre-embryo resulting from the fertilizing of the egg into a uterus connected to the fallopian tube.

2. A method for in vitro fertilization as in claim 1 wherein from the at least one egg harvested, two or more eggs are fertilized and two or more eggs are frozen for future fertilization and wherein the method further comprises shutting down an ovary activity completely and wherein the structure has a material to cause growth of tissue into the fallopian tube.

3. A method for in vitro fertilization as in claim 1 wherein a period of at least one week is elapsed after placing the structure to ensure growth of tissue in the fallopian tube to completely eliminate patency before implanting the pre-embryo into the uterus.

4. A method for in vitro fertilization as in claim 1 wherein the patient or the patient's partner suffers from at least one cause of infertility including hydrosalpinx, endometriosis, a damaged fallopian tube, immunologic factors and other unexplained reasons causing infertility.

5. A method for in vitro fertilization as in claim 1 wherein multiple eggs are harvested in an operation guided by an transvaginal ultrasound probe.

6. A method for in vitro fertilization as in claim 1 wherein the pre-embryo is implanted using a fiber-optic guided catheter with a non-sticking surface coating such as polytetrafluoroethylene (PTFE).

7. A method for in vitro fertilization (IVF) comprising:
performing an IVF protocol for patient preparation and cell retrieval;
placing a structure to occlude at least temporarily a fallopian tube;
fertilizing an egg in vitro from the cell retrieval with a sperm from a donor; and
implanting a fertilized ovum, resulting from the fertilizing of the egg, into a uterus connected to the fallopian tube which is to be occluded or has been occluded by the structure.

8. A method for IVF as in claim 7 wherein the structure occludes the fallopian tube via physical occlusion and wherein the cells which are retrieved in the cell retrieval protocol are oocytes.

9. A method for IVF as in claim 7 wherein the structure in the fallopian tube promotes growth of tissue around and/or through the structure to occlude the fallopian tube.

10. A method for IVF as in claim 7 where in the structure is placed in the fallopian tube by forming the structure in situ in the fallopian tube.

11. A method for IVF as in claim 7 further comprising: verifying occlusion of the fallopian tube before implanting the fertilized ovum.

12. A tubal occlusion device that physically blocks a fallopian tube to reduce the fallopian tube's role in infertility and increase in vitro fertilization (IVF) success.

13. A device as in claim 12 wherein the tubal occlusion device prevents movement of material between a uterine cavity and the fallopian tube and elutes at least one drug to increase success of IVF.

14. A device as in claim 12 wherein the tubal occlusion device promotes tissue growth and tubal occlusion and wherein the tubal occlusion device has a physical size which is designed to engage the walls of a fallopian tube that is enlarged by a disease.

15. A method for performing an in vitro fertilization (IVF), the method comprising of placing a structure in a fallopian tube to at least temporarily treat the fallopian tube;
implanting a fertilized ovum into a uterus coupled to the fallopian tube.

16. The method as in claim 15 wherein the structure treats the fallopian tube by occluding, at least temporarily, the fallopian tube, and wherein the structure is configured to improve the chances for full maturation of the fertilized ovum.

17. The method as in claim 16 wherein the fertilized ovum is obtained by harvesting at least one egg from an ovary of a patient having the uterus and fertilizing the at least one egg with a sperm from a donor and wherein the fertilized ovum is either a single cell or a multi-cellular organism.

18. The method as in claim 17 wherein the structure is placed after the implanting.

19. The method as in claim 17 wherein the structure is placed before the implanting.

20. The method as in claim 17 wherein the structure is placed in the fallopian tube by forming the structure in situ in the fallopian tube.

21. The method as in claim 20 wherein the structure comprises a hydrogel formed in situ in the fallopian tube.

22. The method as in claim 17 wherein the structure is a porous plug and wherein the placing comprises scaring the fallopian tube with at least one of energy or chemicals.

23. The method as in claim 17 wherein the structure is expandable from a first configuration to a second configuration which is radially larger in at least one physical measurement than the first configuration and wherein the structure comprises at least one of a metal and a plastic.

24. The method as in claim 19 further comprising:
verifying occlusion of the fallopian tube before implanting the fertilized ovum, and wherein the verifying comprises at least one of imaging with ultrasound and imaging with x-ray, and imaging with magnetic resonance imaging.

25. The method as in claim 17 further comprising:
verifying placement of the structure in the fallopian tube, wherein the verifying comprises at least one of imaging with an endoscope, imaging with ultrasound, imaging with x-ray, and imaging with magnetic resonance imaging.

26. The method as in claim 17 wherein the structure is removable through the use of a transcervical catheter.

27. The method as in claim 17 wherein the structure is biodegradable such that the fallopian tube is no longer occluded after a period of time.

28. The method as in claim 17 wherein the structure is configured to elute at least one drug to increase tissue ingrowth into the fallopian tube.

29. The method as in claim 17 wherein the structure is configured to elute at least one drug to treat a diseased fallopian tube or uterus.

30. The method as in claim 29 wherein the at least one drug treats at least one of endometriosis, hydorsalpinx, and fibroids.

31. The method as in claim 29 wherein the at least one drug increases the chances for full maturation of the fertilized ovum.

32. The method as in claim 29 wherein the structure comprises at least one of depots to elute the at least one drug and a layer which elutes the at least one drug.

33. The method as in claim 17 wherein the structure has a physical size which is designed to forcibly engage the walls of a fallopian tube is enlarged by a disease.
